(19) United States
(12) Patent Application Publication
(10) Pub. No.: US 2025/0120600 A1
(43) Pub. Date: Apr. 17, 2025

(54) SENSORS FOR MEDICAL ASSEMBLIES, AND ASSOCIATED SYSTEMS AND METHODS

(71) Applicant: Shifamed Holdings, LLC, Campbell, CA (US)

(72) Inventors: Brian Fahey, Menlo Park, CA (US); Peter Andriola, Castro Valley, CA (US); Shahn Sage, Clear Lake, MN (US)

(21) Appl. No.: 18/567,519

(22) PCT Filed: Jun. 17, 2022
(86) PCT No.: PCT/US2022/034027

§ 371 Date: Dec. 6, 2023

Related U.S. Application Data
(60) Provisional application No. 63/211,945, filed on Jun. 17, 2021.

(51) Int. Cl. A61B 5/0215

(52) U.S. Cl. CPC A61B 5/02156

(57) ABSTRACT

The present technology generally relates to a sensor assembly for an implantable medical device. In some embodiments, the sensor assembly includes a housing having a cavity, and a measurement component positioned within the cavity. When the medical device is implanted within a patient, the sensor can be configured to measure one or more physiological parameters of the patient. In some embodiments, the sensor assembly further includes a coupling element positioned within the cavity and at least partially covering, contacting, or encapsulating the measurement component. The coupling element can be configured to transmit or convey the one or more physiological parameters to the measurement component, such that the measurement component can measure the one or more physiological parameters without exposure to an environment external to the sensor assembly.

### CROSS-REFERENCE TO RELATED APPLICATION(S)

This application claims the benefit of U.S. Provisional Patent Application No. 63/211,945, filed Jun. 17, 2021, and incorporated by reference herein in its entirety.

### TECHNICAL FIELD

The present technology generally relates to implantable medical devices and/or medical systems and, in particular, to sensors for implantable medical devices and/or systems.

### BACKGROUND

Implantable devices and systems are utilized in modern medicine to provide a host of diagnostic and/or therapeutic benefits. For example, implantable shunting systems are widely used to treat a variety of patient conditions by shunting fluid from a first body region/cavity to a second body region/cavity. The flow of fluid through the shunting systems is primarily controlled by the pressure gradient across the shunt lumen and the geometry (e.g., size) of the shunt lumen. One challenge with conventional shunting systems is selecting the appropriate geometry of the shunt lumen for a particular patient. A lumen that is too small may not provide enough therapy to the patient, while a lumen that is too large may create new issues in the patient. Despite this, most conventional shunts cannot be adjusted once they have been implanted. Accordingly, once the system is implanted, the therapy provided by the shunting system cannot be adjusted or titrated to meet the patient's individual needs.

Some implantable medical systems use sensors to measure physiological parameters (e.g., a shunt device that includes sensors to measure parameters in the first body region and/or the second body region). Oftentimes, electrical components of sensors must be fluidically-isolated from anatomical regions in order to remain functional or accurate, and as such can be integrated into sealed (e.g., hermetically-sealed) housings, casings, and the like. For housings associated with pressure sensors, the housings generally can have a pressure responsive element at or near a surface that is affixed to the housing in a manner that is impermeable to fluid ingress. This pressure responsive element can be, for example, a diaphragm whose deflection profile changes in response to changes in local physiological pressure. If the pressure responsive element is comprised of a material with suitable mechanical properties (e.g., flexural rigidity), it will sufficiently transfer pressure signals from outside of the housing to the interior of the housing without meaningful artifact (e.g., damping).

Challenges associated with systems described in the prior art can be related to manufacturing of appropriate sensor housings and related components. These challenges can include, for example, ensuring adequate coupling of a signal from outside of a housing to the electrical and/or mechanical components located internal to the housing that are sensitive to the signal (i.e., the “measurement component(s)” of the sensor apparatus). This coupling is generally accomplished using a coupling element, as elaborated upon below. Some approaches to pressure sensor assemblies described in the prior art have attempted to use a combined material as both a pressure responsive fluid-sealing element and as a pressure coupling element. These approaches, however, have been associated with limited success, as fluid ingress or diffusion over time can lead to degraded performance. Other approaches in the prior art use a separate diaphragm as a pressure responsive element and a fluid such as silicone oil as a coupling element that relays pressure signals produced in an external anatomical region (e.g., in the first body region or the second body region) to the measurement components within a housing. However, these solutions are often costly and difficult to manufacture and have been associated with unreliable performance, for example due to leaking of fluid out of the housing due to imperfections in the seal. Further, as sensor assemblies become smaller (e.g., to be compatible with percutaneously-delivered medical devices), the manufacturing difficulties associated with proper injection and/or encapsulation of a fluid coupling element escalate considerably. There is therefore a need for improved sensor assemblies for use in medical implants.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1 is a schematic illustration of an interatrial shunting system including one or more sensors and configured in accordance with select embodiments of the present technology.

FIG. 2A is a perspective view of a sensor assembly configured in accordance with select embodiments of the present technology.

FIG. 2B is a front view of the sensor assembly of FIG. 2A.

FIG. 2C is a top view of the sensor assembly of FIG. 2A.

FIG. 2D is a front view of the sensor assembly of FIG. 2A including a pressure responsive element configured in accordance with select embodiments of the present technology.

### DETAILED DESCRIPTION

The present technology is generally directed to medical systems including one or more sensors. In some embodiments, for example, the system includes a shunting element implantable into a patient at or adjacent to a septal wall. The shunting element, for example, can fluidly connect a left atrium and a right atrium of the patient to facilitate blood flow therebetween. In some embodiments, a size and/or shape of the shunting element is adjustable to alter the flow through the shunt. In some embodiments, the shunting system can be configured to adjust the size and/or shape of the shunting element in response to a sensed (e.g., measured, detected, etc.) physiological parameter. For example, the system can include one or more sensors configured to sense a physiological parameter (e.g., temperature, pressure, etc.) in a first body chamber (e.g., the left atrium) and/or in a second body chamber (e.g., the right atrium), and the shunting element can be adjusted at least in part based on the sensed physiological parameter. In some embodiments, the sensors can include one or more capacitive sensors, piezoelectric sensors, piezoresistive sensors, Micro-electromechanical System (“MEMS”) sensors, and/or any other suitable sensor.

In some embodiments, a sensor includes a housing or casing that is substantially impermeable to fluid. In some embodiments, one or more coupling elements can be positioned at least partially between one or more measurement components of the sensor and the exterior of the housing that interfaces with an anatomical region (e.g., the first body chamber and/or the second body chamber). In some embodiments, the housing further includes a pressure responsive element (e.g., a diaphragm) between the measurement component(s) and an anatomical region. In embodiments, a coupling element may be chosen to be best suited for the parameter a sensor is designed to measure (e.g., temperature, pressure, etc.). The housing, pressure responsive element, and/or coupling element can at least partially insulate or isolate the one or more measurement components from the anatomical region, while still allowing the one or more measurement components to sense physiological parameters. For example, the pressure responsive element and a pressure coupling element can be configured to work in tandem to transmit a pressure (e.g., a left atrial pressure) from the anatomical region to the one or more measurement components. In embodiments, the pressure coupling element can be an elastomeric material such as polydimethylsiloxane (PDMS) or another similar (e.g., silicone) polymer. It is expected that medical systems including sensors that implement housings, pressure responsive elements, and/or pressure coupling elements as described herein can reduce or prevent fluids (e.g., blood) in the first and/or second body cavities from disrupting or interfering with the operation of the one or more sensors. As explained in detail below, the disclosed sensor designs are further expected to improve the simplicity and lower the costs associated with manufacturing medical sensors, while providing assemblies that are expected to be both more accurate and robust in clinical settings.

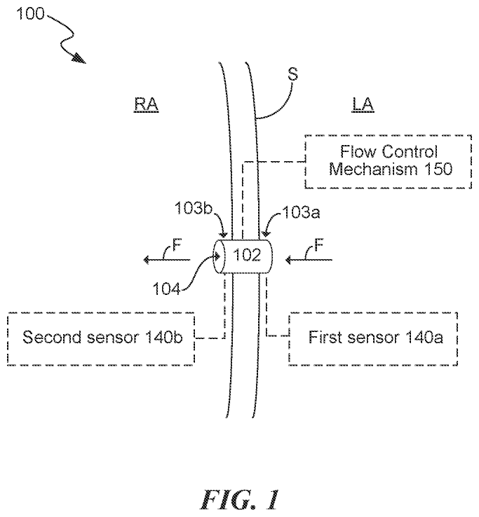

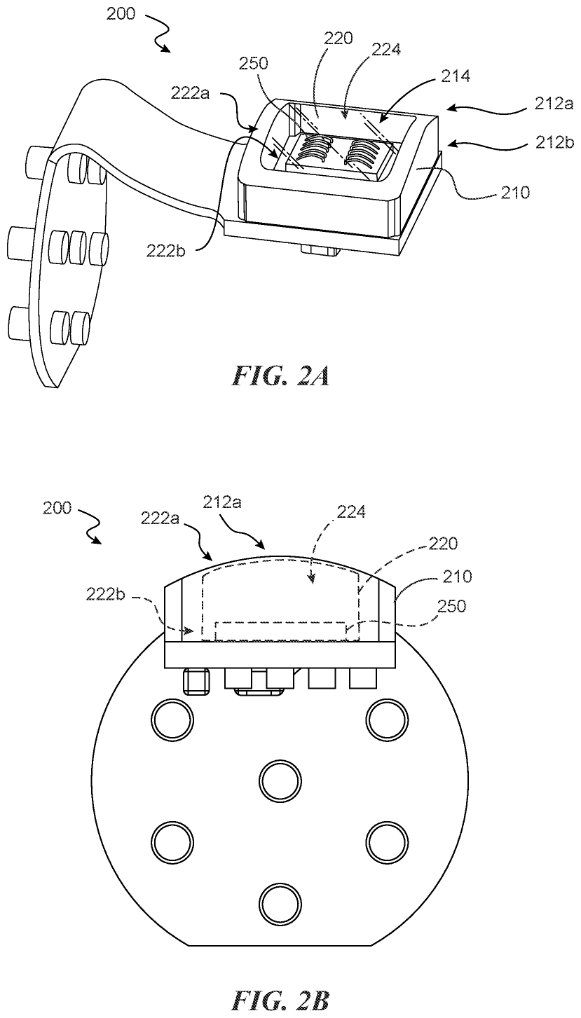

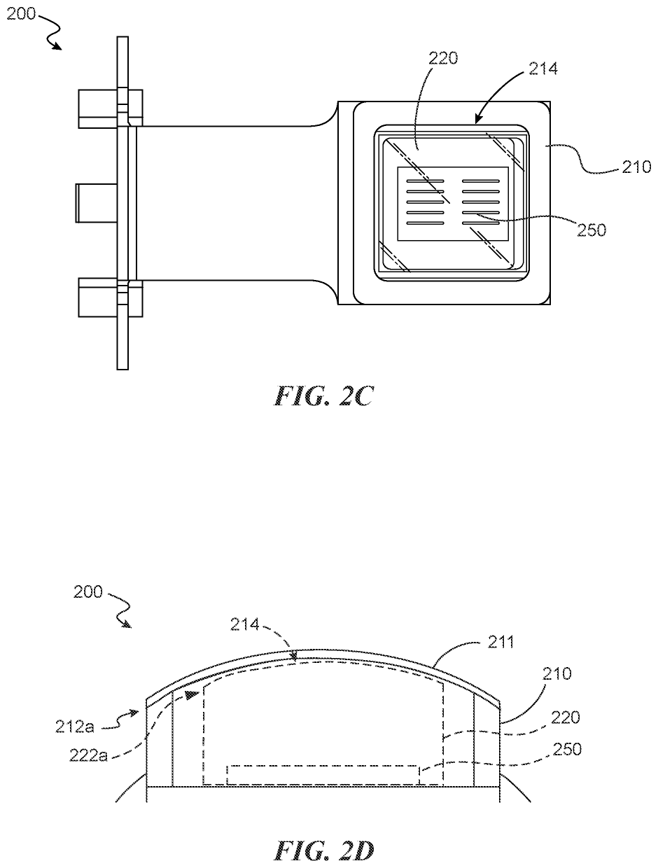

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1-2D.

Reference throughout this specification to “one embodiment” or “an embodiment” means that a particular feature, structure or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “substantially,” “approximately,” and “about” are used herein to mean the stated value plus or minus 10%.

As used herein, the terms “interatrial device”, “interatrial shunt device”, “IAD”, “IASD”, “interatrial shunt”, and “shunt” are used interchangeably to refer to a device that, in at least one configuration, includes a shunting element that provides a blood flow between a first chamber (e.g., a left atrium of a heart) and a second chamber (e.g., a right atrium or coronary sinus of the heart) of a patient. Although described in terms of a shunt between the atria, namely the left and right atria, one will appreciate that the technology may be applied equally to devices positioned between other chambers and passages of the heart, between other parts of the cardiovascular system, or between other parts of the body. For example, any of the shunts described herein, including those referred to as “interatrial,” may nevertheless be used and/or modified to shunt between the left atria and the coronary sinus, or between the right pulmonary vein and the superior vena cava. Moreover, while the disclosure herein primarily describes medical devices for shunting blood in the heart, the present technology can be readily adapted for medical devices used to shunt other fluids—for example, devices used for aqueous shunting or cerebrospinal fluid shunting. The present technology may also be adapted to a variety of implanted medical devices in addition to shunts. The headings provided herein are for convenience only and do not interpret the scope or meaning of the claimed present technology.

### A. Medical Systems Including Sensor(s)

FIG. 1 is a schematic illustration of an interatrial shunting system 100 (“system 100”) configured in accordance with embodiments of the present technology. The system 100 includes a shunting element 102 defining a lumen 104 therethrough. The shunting element 102 can include a first end portion 103a positioned in a left atrium LA of a patient and a second end portion 103b positioned in a right atrium RA of the patient. Accordingly, when implanted in the septal wall S, the system 100 fluidly connects the left atrium LA and the right atrium RA via the lumen 104. When the system 100 is implanted within the patient to treat heart failure, blood generally flows through the lumen 104 in flow direction F (i.e., from the left atrium LA to the right atrium RA).

The system 100 can also include one or more sensors 140. For example, the system 100 can include a first sensor 140a positionable within the left atrium LA and a second sensor 140b positionable within the right atrium RA (collectively referred to as sensors 140). Further details regarding sensors configured for use with the system 100 or other medical systems are provided below with reference to FIGS. 2A-2D. The sensors 140 can measure one or more physiologic parameters related to the system 100 or the environment proximate to the sensors 140. For example, the first sensor 140a can be configured to measure left atrium LA pressure and the second sensor 140b can be configured to measure right atrium RA pressure. In some embodiments, the system 100 can further include a processor (not shown) configured to calculate a pressure differential between the left atrium LA and the right atrium RA, or other calculated parameters, based on the information measured by the sensor(s) 140. As described below, the system 100 may be adjusted based on the parameters measured by the sensor(s) 140 and/or the pressure differential calculated by the processor. Additional features of some embodiments of the sensors 140 useful in the system described herein are disclosed in International PCT Application No. PCT/US20/63360, the disclosure of which is incorporated herein by reference in its entirety.

The system 100 also includes a flow control mechanism 150 (e.g., an actuation mechanism). The flow control mechanism 150 is configured to change a size, shape, or other characteristic of the shunting element 102 to change the flow of fluid through the lumen 104. In some embodiments, the flow control mechanism 150 can selectively change a size and/or shape of the lumen 104 to change the flow through the lumen 104. For example, the flow control mechanism 150 can be configured to selectively increase a diameter of the lumen 104 and/or selectively decrease a diameter of the lumen 104 in response to an input. In other embodiments, the flow control mechanism 150 is configured to otherwise affect a shape of the lumen 104. Accordingly, the flow control mechanism 150 can be coupled to the shunting element 102 and/or can be included within the shunting element 102. For example, in some embodiments the flow control mechanism 150 is part of the shunting element 102 and at least partially defines the lumen 104. In other embodiments, the flow control mechanism 150 is spaced apart from but operably coupled to the shunting element 102.

In some embodiments, at least a portion of the flow control mechanism 150 can comprise a shape memory material. The shape memory portion can include nitinol, a shape memory polymer, a pH-based shape memory material, or any other suitable material configured to move or otherwise adjust as would be understood by one of skill from the description herein. The shape memory portion can be characterized by a curve that defines the amount of deformation the portion undergoes in response to a particular input (e.g., an applied stress). For example, the flow control mechanism 150 can include nitinol element that is configured to change shape in response to exposure to energy, such as light and/or heat. In such embodiments, the flow control mechanism 150 can be selectively actuated by applying energy directly or indirectly to the nitinol element. Additional embodiments of flow control mechanisms suitable for use with the present technology are described in International PCT Application No. PCT/US20/38549, the disclosures of which is incorporated herein by reference in its entirety, and International PCT Application No. PCT/US20/63360, the disclosure of which was previously incorporated by reference.

In some embodiments, the flow control mechanism 150 is coupled to a processor (not shown) that calculates the pressure differential between the left atrium and right atrium based, at least in part, on the measurements taken by the sensors 140. If the calculated pressure differential falls outside of a predetermined range, the processor can direct the flow control mechanism 150 to change the flow through the shunting element 102.

FIGS. 2A-2D illustrate various views of a sensor subassembly 200 (“subassembly 200”) in accordance with select embodiments of the present technology. The subassembly 200 can be part of a stand-alone sensing system, part of an interatrial shunting system such as the system 100 of FIG. 1, or part of other suitable systems configured for implantation within a patient. In some embodiments, for example, the subassembly 200 can be generally similar to, the same as, or included in (e.g., a component of) the first sensor 140a and/or the second sensor 140b of FIG. 1.

Referring first to FIG. 2A, the subassembly 200 includes a housing 210 having a first (e.g., upper) end portion 212a and a second (e.g., lower) end portion 212b. The housing 210 can define a cavity 214, and the cavity 214 can be configured to receive and house one or more sensor measurement component(s) 250. For example, the subassembly 200 can be a component of a sensor assembly that is positioned within a left atrium LA or a right atrium RA of a patient's heart (e.g., such as the sensors 140a, 140b of the system 100 described above with reference to FIG. 1) and can measure one or more physiologic parameters related to the environment proximate to the sensor assembly. The measurement component(s) 250, for example, can be a MEMS-based sensor configured to measure a pressure in the left atrium or the right atrium, and/or any other suitable sensor or sensing element configured to measure one or more physiologic parameters of the patient.

FIG. 2B is a front view of the subassembly 200. Referring to FIGS. 2A and 2B together, the subassembly 200 can further include a coupling element 220 positioned within the cavity 214. Accordingly, the coupling element 220 can at least partially cover, contact, encapsulate, or otherwise interface with the measurement component(s) 250, e.g., to assist with conveying signals to the measurement component(s) 250. The coupling element 220 can be configured to transmit or convey one or more physiological parameters from the environment (e.g., left atrium, right atrium, etc.) external to the subassembly 200 to the measurement component(s) 250 so that the measurement component(s) 250 can sense the one or more physiological parameters. In the illustrated embodiment, for example, the coupling element 220 has a first (e.g., upper) end portion 222a and a second (e.g., lower) end portion 222b. The first end portion 222a can be positioned proximate to and/or configured to interface (directly or indirectly) with an environment external to the subassembly 200 (e.g., within a desired body chamber and/or anatomical region), and the second end portion 222b can at least partially contact (and, in some embodiments, be mechanically coupled to) the measurement component(s) 250. In some embodiments, the first end portion 222a of the coupling element 220 interfaces with the measurement component(s) 250, or both portions 222a-b of the coupling element 220 interface with measurement component(s) 250. In some embodiments, the coupling element 220 is roughly uniform in shape and/or distribution and, therefore, is not associated with first and second end portions. In some embodiments, the environment external to the subassembly 200 can exert a force or pressure (either directly or indirectly, e.g., via a deflection of a pressure responsive element) on the first end portion 222a of the coupling element 220, and the force or pressure can be transmitted by the coupling element 220 to the measurement component(s) 250 such that the measurement component(s) 250 can measure the force or pressure in the desired anatomical region.

Some embodiments may include multiple coupling elements 220 that work in tandem to couple information regarding physiological parameters from parameter sensitive element(s) (e.g., pressure responsive element(s)) to measurement component(s) 250). In such embodiments, for example, a first coupling element may interface directly with a parameter sensitive element and a second coupling element may interface with a measurement component. The first and second coupling elements are operably coupled to one another such that, in practice, they function similar to a single coupling element. In embodiments including multiple coupling elements, the different coupling elements can be comprised of similar or differing materials, and can be manufactured to have similar or different mechanical properties (e.g., durometer, shear modulus, etc.).

The coupling element 220 can be formed from a transmissive material 224. The transmissive material 224 can be an elastomeric material, for example a silicone elastomer (such as polydimethylsiloxane, PDMS), or another suitable material. The transmissive material 224 can have a high bulk modulus, such that the transmissive material 224 is generally resistant to compression, e.g., in response to a pressure in the environment external to the subassembly 200. For example, the transmissive material 224 can have a bulk modulus K of at least 0.5 megapascals (MPa), 1 MPA, 2 MPA, 5 MPA, 10 MPa, 20 MPA, 50 MPA 100 MPa, 1 gigapascals (GPa), 2 GPa or any amount therebetween. In some embodiments, the transmissive material 224 can have a hardness, as measured using the Shore hardness scale, of at least OO0, OO50, OO100, A0, A10, A20, A30, A40, A50, and/or any amount therebetween.

The bulk modulus of silicone elastomers (such as PDMS) is typically less than the bulk modulus of the silicone oils used in conventional (e.g., larger, non-medical) sensor assemblies described in the prior art. Accordingly, one skilled in the art may generally expect that silicone elastomers have reduced viability as coupling elements and/or transmissive materials due to this difference in compressibility. Further, one could expect the use of a solid material coupling element (as opposed to a fluid or gas) could have further detrimental impact on the viability of the sensors due to challenges related to the coupling of solid components with measurement components. However, as described in more detail below, sensors configured in accordance with the present technology overcome these challenges and enable a silicone elastomer material to be utilized as a transmissive material without affecting the performance and/or accuracy of the sensors.

An unexpected finding associated with embodiments of the present technology configured to measure a pressure signal is that when used in conjunction with a very thin, low flexural rigidity pressure responsive element (e.g., a 10 μm-50 μm titanium diaphragm), an elastomeric coupling element with a relatively lower bulk modulus (compared to a silicone oil) can effectively convey a pressure signal accurately from a region external to a housing (e.g., an anatomical region such as a heart chamber) to a measurement component internal to a housing. This particular combination of pressure responsive elements and pressure coupling elements has not been described in the prior art, as the need for such a design is reduced outside of the realm of small, hermetically-sealed sensors intended for percutaneous medical implantation.

As described above, a further challenge to the viability of sensors using solid (e.g., elastomeric) coupling elements is an ability to manufacture such a component in a way that enables reliable functionality. More specifically, it can be difficult to ensure that a solid coupling element makes sufficient and intimate contact with measurement component(s) of a sensor apparatus without introducing voids, gas bubbles, and/or other irregularities or disturbances that can introduce biases, artifacts, and/or other unwanted degradations to a conveyed signal. As disclosed in more detail below, the present technology overcomes this challenge with a novel manufacturing method aimed at depositing a solid coupling element in a manner such that it can cover, contact, or encapsulate a measurement component without voids, gas bubbles, and/or other irregularities.

In embodiments in which the transmissive material 224 is composed of a silicone elastomer (such as PDMS), the coupling element 220 can be formed by depositing (e.g., pouring) the transmissive material 224 into the cavity 214 when the transmissive material 224 is in a liquid state where it has not yet been cured, hardened, etc., and allowing the transmissive material 224 to cure or harden for a predetermined amount of time. For example, the predetermined amount of time can be at least 1 minute, 5 minutes, 10 minutes, 20 minutes, 30 minutes, 45 minutes, 1 hour, 1 day, or any other amount of time. Such a method enables the transmissive material 224, while in its liquid state, to completely wet and form an intimate, void-free contact with the measurement component(s) 250. Such a method also enables this intimate, void-free contact to be preserved after the transmissive material 224 cures or hardens to form the coupling element 220. If the transmissive material 224/coupling element 220 was to be applied in a cured or hardened state, said intimate contact would not be formed, and any dimensional differences between the mating surface of the cured or hardened coupling element 220 and the mating surface of the measurement component(s) 250 would form voids. Maintaining such void-free contact is important to the pressure transmitting function of the coupling element 220 as otherwise pressure applied to the coupling element 220 (e.g., by a pressure responsive element) would simply force the coupling element 220 into the void rather than conveying the pressure to the measurement component(s) 250. In other embodiments, however, the coupling element 220 can be formed using other suitable processes or techniques known to one of skill in the art.

As best seen in FIG. 2B, the first end portion 222a of coupling element 220 (shown in dashed line) can correspond to, be mechanically-coupled to, be integrated with, or otherwise interface with the first end portion 212a of the housing 210, such that the respective first end portions 212a, 222a can form a generally coupled and/or evenly interfaced surface. For example, in the illustrated embodiment the first end portion 212a of the housing 210 has a generally curved or arcuate shape, and the first end portion 222a of the coupling element 220 can have a correspondingly curved or arcuate shape, allowing for enhanced coupling of signals from one structure to the other. As described previously regarding FIG. 2A, at least a portion of the coupling element 220 can at least partially contact and/or surround the measurement component(s) 250 (shown in dashed line in FIG. 2B). In some embodiments, and as described in greater detail below and with reference to FIG. 2D, a pressure responsive element 211 (FIG. 2D) can be attached to, coupled to, or otherwise integrated with at least part of the first end portion 212a of the housing 210.

In the illustrated embodiment, the first end portion 212a of the housing 210 is shown slightly spaced apart from the first end portion 222a of the coupling element 220 to more clearly show the geometry of both portions. As one skilled in the art will appreciate, however, in other embodiments, the first end portion 212a of the housing 210 can be aligned with, attached to, interfaced with, or otherwise more closely integrated to the coupling element 220.

FIG. 2C is a top view of the subassembly 200. As shown, in the illustrated embodiment the housing 210, cavity 214, and coupling element 220 have substantially square shapes that are generally similar/matching. In other embodiments, however, the housing 210, cavity 214, and/or the coupling element 220 can each have a same or a different shape. For example, in some embodiments the housing 210, cavity 214, and/or the coupling element 220 can have a rectilinear, circular, triangular, pentagonal, hexagonal, or any other suitable shape. In some embodiments, the coupling element 220 can have a shape corresponding to the shape of the cavity 214. For example, during a manufacturing process, the transmissive material 224 can at least partially fill the cavity 214 such that the coupling element 220 assumes a shape generally similar to the same as the shape of the cavity 214.

Additionally, in the illustrated embodiment, each of the housing 210, cavity 214, and coupling element 220 have rounded corners, which may be beneficial for reducing stress concentration or for other purposes. In other embodiments, however, the housing 210, cavity 214, and/or coupling element 220 can have sharp corners.

FIG. 2D is a front view of the housing 210 with certain aspects of the subassembly 200 omitted for the purpose of clarity. In some embodiments, the first end portion 212a of the housing 210 can include or be otherwise integrated (e.g., attached, coupled, etc.) with a pressure responsive element 211. In some embodiments, the first end portion 212a of the housing 210 can have an opening or open section (not shown) where the pressure responsive element 211 can mechanically or functionally interface directly with a coupling element 220. In the illustrated embodiment, for example, the cavity 214 of the housing can be configured such that the pressure responsive element 211 can be coupled (e.g., mechanically, communicatively, etc.) to the first end portion 222a of the coupling element 220. The pressure responsive element 211 can be configured to transmit or convey the one or more physiological parameters from the environment external to the subassembly 200 (e.g., the anatomical region, the left atrium LA, the right atrium RA, etc.) to the measurement component(s) 250, e.g., via the coupling element 220 as described previously herein with reference to FIGS. 2A-2C. The pressure responsive element 211 can be or can include a diaphragm formed from a material such a metal (e.g., titanium), a composite, polymer, a combination thereof, and/or any other suitable material. In at least some embodiments, for example, the pressure responsive element 211 can include a thin titanium diaphragm as described previously.

Sensors including coupling elements configured in accordance with embodiments of the present technology represent an improvement over traditional sensors that include silicone oil, or other fluids. For example, the subassembly 200 with the coupling element 220 over the measurement component(s) 250 can be less costly to produce and easier to manufacture than traditional sensors. Further, as noted previously, sensor assemblies configured in accordance with the present technology are expected to be both more accurate and robust in clinical settings than conventional devices.

### B. Examples

Several aspects of the present technology are set forth in the following examples:

1. A sensor assembly for an implantable medical device, the sensor assembly comprising:
a housing having a cavity;
a measurement component positioned within the cavity, wherein, when the medical device is implanted within a patient, the measurement component is configured to measure one or more physiological parameters of the patient; and
a coupling element positioned within the cavity and at least partially covering, contacting, and/or encapsulating the measurement component, wherein the coupling element is composed of an elastomeric material.

2. The sensor assembly of example 1 wherein the measurement component is a MEMS sensor.

3. The sensor assembly of example 1 or example 2 wherein the one or more physiological parameters include a left atrial pressure and/or a right atrial pressure of the patient.

4. The sensor assembly of any one of examples 1-3 wherein the elastomeric material comprises polydimethylsiloxane.

5. The sensor assembly of any one of examples 1-4 wherein the elastomeric material has a bulk modulus of at least 0.5 MPa

6. The sensor assembly of any one of examples 1-4 wherein the elastomeric material has a bulk modulus of at least 100 MPa.

7. The sensor assembly of any one of examples 1-6 wherein the elastomeric material has a Shore hardness of A0.

8 The sensor assembly of any one of examples 1-6 wherein the elastomeric material has a Shore hardness between A0 and A50.

9. The sensor assembly of any one of examples 1-8 wherein, when the medical device is implanted within the patient, the coupling element is positioned to transmit pressure from a region proximate to the sensor assembly to the sensor.

10. The sensor assembly of any one of examples 1-9 wherein the medical device comprises an interatrial shunt configured to be implanted within a heart of the patient.

11. The sensor assembly of any one of examples 1-10, further comprising a pressure responsive element at least partially coupled to a first end portion of the housing and/or a second end portion of the coupling element.

12. A method of manufacturing a sensor assembly for an implantable medical device, the method comprising:
providing a housing having a cavity;
positioning a measurement component within the cavity; and
depositing a transmissive material within the cavity to form a coupling element, wherein the coupling element at least partially covers, contacts, and/or encapsulates the measurement component.

13. The method of example 12 wherein transmissive material is an elastomeric material or a silicone elastomer.

14. The method of example 13 wherein the transmissive material is polydimethylsiloxane.

15. The method of any one of examples 12-14 wherein the transmissive material has a bulk modulus of at least 0.5 MPa.

16. The method of any one of examples 12-15 wherein the transmissive material has a Shore hardness between A0 and A50.

17. The method of any one of examples 12-16 wherein the transmissive material is in a liquid or generally unhardened state when deposited into the cavity.

18. The method of any one of examples 12-17 wherein depositing the transmissive material includes allowing the transmissive material to cure and/or harden for a predetermined amount of time.

19. The method of example 18 wherein the predetermined amount of time is least one minute.

20. The method of example 18 wherein the predetermined amount of time is between one minute and one day.

21. The method of any one of examples 12-20 wherein, when the medical device is implanted within a patient, the sensor is configured to measure one or more physiological parameters of the patient.

### CONCLUSION

Embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, WiFi, or other protocols known in the art; energy harvesting means, for example a coil or antenna which is capable of receiving and/or reading an externally-provided signal which may be used to power the device, charge a battery, initiate a reading from a sensor, or for other purposes. Embodiments may also include one or more sensors, such as pressure sensors, impedance sensors, accelerometers, force/strain sensors, temperature sensors, flow sensors, optical sensors, cameras, microphones or other acoustic sensors, ultrasonic sensors, ECG or other cardiac rhythm sensors, SpO₂ and other sensors adapted to measure tissue and/or blood gas levels, blood volume sensors, and other sensors known to those who are skilled in the art. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe devices that are generally described as being used to create a path of fluid communication between the left atrium and right atrium, the left ventricle and the right ventricle, or the left atrium and the coronary sinus, it should be appreciated that similar embodiments could be utilized for shunts between other chambers of heart or for shunts in other regions of the body.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A sensor assembly for an implantable medical device, the sensor assembly comprising:
a housing having a cavity;
a measurement component positioned within the cavity, wherein, when the medical device is implanted within a patient, the measurement component is configured to measure one or more physiological parameters of the patient; and
a coupling element positioned within the cavity and at least partially covering, contacting, and/or encapsulating the measurement component, wherein the coupling element is composed of an elastomeric material.

2. The sensor assembly of claim 1 wherein the measurement component is a MEMS sensor.

3. The sensor assembly of claim 1 wherein the one or more physiological parameters include a left atrial pressure and/or a right atrial pressure of the patient.

4. The sensor assembly of claim 1 wherein the elastomeric material comprises polydimethylsiloxane.

5. The sensor assembly of claim 1 wherein the elastomeric material has a bulk modulus of at least 0.5 MPa

6. The sensor assembly of claim 1 wherein the elastomeric material has a bulk modulus of at least 100 MPa.

7. The sensor assembly of claim 1 wherein the elastomeric material has a Shore hardness of A0.

8. The sensor assembly of claim 1 wherein the elastomeric material has a Shore hardness between A0 and A50.

9. The sensor assembly of claim 1 wherein, when the medical device is implanted within the patient, the coupling element is positioned to transmit pressure from a region proximate to the sensor assembly to the sensor.

10. The sensor assembly of claim 1 wherein the medical device comprises an interatrial shunt configured to be implanted within a heart of the patient.

11. The sensor assembly of claim 1, further comprising a pressure responsive element at least partially coupled to a first end portion of the housing and/or a second end portion of the coupling element.

12. A method of manufacturing a sensor assembly for an implantable medical device, the method comprising:
providing a housing having a cavity;
positioning a measurement component within the cavity; and
depositing a transmissive material within the cavity to form a coupling element, wherein the coupling element at least partially covers, contacts, and/or encapsulates the measurement component.

13. The method of claim 12 wherein transmissive material is an elastomeric material or a silicone elastomer.

14. The method of claim 12 wherein the transmissive material is polydimethylsiloxane.

15. The method of claim 12 wherein the transmissive material has a bulk modulus of at least 0.5 MPa.

16. The method of claim 12 wherein the transmissive material has a Shore hardness between A0 and A50.

17. The method of claim 12 wherein the transmissive material is in a liquid or generally unhardened state when deposited into the cavity.

18. The method of claim 12 wherein depositing the transmissive material includes allowing the transmissive material to cure and/or harden for a predetermined amount of time.

19. The method of claim 18 wherein the predetermined amount of time is least one minute.

20. The method of claim 18 wherein the predetermined amount of time is between one minute and one day.

21. The method of claim 12 wherein, when the medical device is implanted within a patient, the sensor is configured to measure one or more physiological parameters of the patient.
